(19) United States
(12) Patent Application Publication
(10) Pub. No.: US 2023/0335234 A1
(43) Pub. Date: Oct. 19, 2023

(54) System and Method for Real-Time Reconciliation and Reformulation of Integrative Health and Wellness Treatments

(71) Applicant: Nima Veiseh, Grand Haven, MI (US)

(72) Inventor: Nima Veiseh, Grand Haven, MI (US)

(21) Appl. No.: 18/301,877

(22) Filed: Apr. 17, 2023

Related U.S. Application Data
(60) Provisional application No. 63/331,657, filed on Apr. 15, 2022.

(51) Int. Cl. G16H 10/20

(52) U.S. Cl. CPC G16H 10/20

(57) ABSTRACT

Disclosed is a system and method real-time reconciliation and reformulation of integrative health and wellness treatments.

The current application claims a priority to the U.S. Provisional Pat. Application Serial No. 63/331,666 filed on Apr. 15, 2022. The current application is filed on Apr. 17, 2023 while Apr. 15, 2023 was on a weekend.

### FIELD OF THE INVENTION

The present invention relates generally to a method for an software application (app). More specifically, the present invention is a method for an app that serves as a nexus to provide a means of accessing platforms.

### BACKGROUND OF THE INVENTION

In the modern age of the Internet, the supply chain of goods and information are becoming largely indistinguishable. This invention is an appliance that serves as a novel nexus of these two systems to provide a means of accessing platforms as well as maintaining the security and integrity of the interactions between wellness providers and consumers, to facilitate care that includes, but is not limited to, integrative, alternative and holistic health and/or wellness treatments.

### BRIEF DESCRIPTION OF THE DRAWINGS

FIG. 1 is a diagram of the present invention; and

FIG. 2 is another diagram of the present invention.

### DETAIL DESCRIPTIONS OF THE INVENTION

All illustrations of the drawings are for the purpose of describing selected versions of the present invention and are not intended to limit the scope of the present invention.

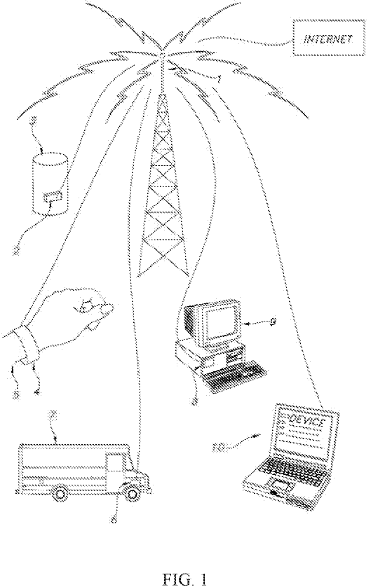

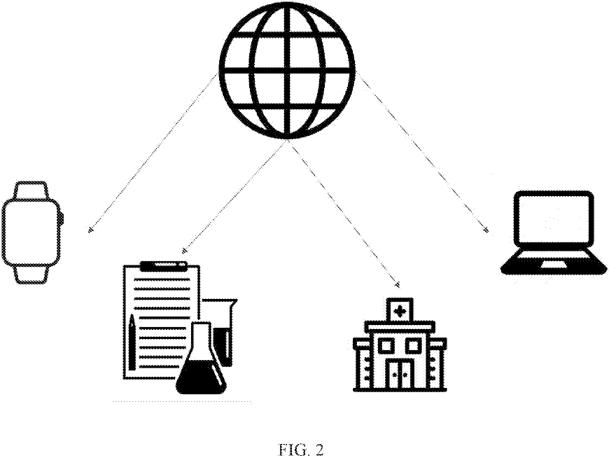

As can be seen in FIGS. 1 and 2, the present invention is an augmented telehealth experience to enable a new era in therapeutic treatments. The new telehealth appliance must solve a number of issues that have been traditionally part of the past medical history. 1, the reach of the hospital is extending more deeply into the home where the home is not just the place for monitoring, but also the administration (or reconciliation) of treatments. This hybrid approach requires the utilization of a dynamic intelligent health (or wellness) record (IHR or IWR) to serve as a digital nexus between the dynamically changing settings along the patient journey. The intelligence of the record is derived from its functionality to track the changing contexts and settings, both along where treatments are derived along their supply chains, as well as the administration setting that ultimately leads to bespoke outcomes. This is very important in the use of psychological treatments because the efficacy of the treatments is related to the setting in which the person is receiving said treatments. Thus, for the first time in medical history the tool actualizing the treatment and how therapy are administered must account for the context of the personal setting of the patient because it affects the overall efficacy of the application and administration of that treatment. This invention helps to reduce the endogeneity, as well as the observed noise-to-signal ratio, of clinical observation and administration, along the patient journey. A major application of this patent is that the present inventor has engineered continuity management so that in case someone is going through a psychedelic “trip” and there is a disconnection, the patient does not need to worry about feeling alone or lost as the application will automatically detect and reconnect. This appliance connects through an IoT (Internet of Things) device to enable cooperative monitoring and feedback loop into the appliance. The device is blockchain-enabled so individuals do not need to worry about their privacy or the integrity of their medical or wellness records, thus further reducing the noise-to-signal ratio of clinical administration; capitalizing on the effects of Decenomics, or Decentralized Economics, at scale. They can retain continuity of treatment as well the necessarily required piece of mind that may be a prerequisite for the treatment to be administered. This feedback loop then creates an opportunity for intelligent and informed reformulation or reconstitution of the treatment and its administration, for the goal of the continuous improvement of subject outcomes.

A key component is that the appliance can work as an independent device, tolerant of intermittencies, on or off internet connectivity, that is more portable and less subject to the biases and scrutiny introduced by an irregular regulatory environment. Portability is key because clinical, laboratory or onsite environments may be required for the data intake in order to maximize the integrity of that data. Such data intake can result in real time data feedback to clinicians, treatment developers and manufactures, the opportunity to execute distributed clinical trials without the pains of existing infrastructure/on-site requirements and/or the activation of a citizen scientist ecosystem.

The prior art for the present invention includes the following:
1. US8631126B2
2. WO2006091528A3
3. WO2009110991A2
4. US20220013202A1
5. “Markets With Memory”, by Nima Veiseh, 2019.ISBN: 9781700165121
6. US10419463B2
7. US10693900B2
8. US20200201850A1
9. US11023539B2

Although the invention has been explained in relation to its preferred embodiment, it is to be understood that many other possible modifications and variations can be made without departing from the spirit and scope of the invention.

What is claimed is:

1. A method real-time reconciliation and reformulation of integrative health and wellness treatments, the method comprising the steps of:
(A) providing a browser that can be viewed as a website or viewed through a software application managed by a portable computing device;
(B) executing a merger between, but not limited to, the supply chain and medical legs of a clinical support structure in the computer architecture of the model;
(C) hosting a portal clinic opportunity; and
(D) executing distributed clinical trials.
